(19) United States
(12) Patent Application Publication
(10) Pub. No.: US 2022/0273430 A1
(43) Pub. Date: Sep. 1, 2022

(54) TRANSCATHETER PAPILLARY MUSCLE BAND

(71) Applicant: Cardiac Success Ltd., Yokneam (IL)

(72) Inventors: David Maier NEUSTADTER, Nof Ayalong (IL); Boaz MANASH, Yaakow (IL)

(73) Assignee: Cardiac Success Ltd., Yokneam (IL)

(21) Appl. No.: 17/634,450

(22) PCT Filed: Sep. 17, 2020
(86) PCT No.: PCT/IB2020/058687

§ 371 Date: Feb. 10, 2022

Related U.S. Application Data
(60) Provisional application No. 62/901,844, filed on Sep. 18, 2019.

(51) Int. Cl. A61F 2/24; A61B 17/12

(52) U.S. Cl. CPC A61F 2/2442; A61B 17/12013; A61B 2017/00243

(57) ABSTRACT

Embodiments of the present disclosure include a device for repositioning papillary muscles, including a band configured to at least partially surround a cluster of papillary muscles. The cluster of papillary muscles may have an outer peripheral boundary, and the band may include a first end and a second end. The device for repositioning papillary muscles may further include a winch coupled to a location proximate the first end of the band. The winch may be configured to adjust a length of the band to apposition the papillary muscles in the cluster.

### CROSS-REFERENCE TO RELATED APPLICATIONS

This application claims priority from U.S. Provisional Patent Application No. 62/901,844, filed Sep. 18, 2019, which is hereby incorporated by reference in its entirety in the present application.

### TECHNICAL FIELD

Some applications of the present invention relate in general to devices and methods for transcatheter navigation to a ventricle of a heart. More specifically, some applications of the present invention relate to devices and methods of transcatheterly delivering and navigating a band to at least partially surround a plurality of papillary muscles in a heart of a body and appositioning the papillary muscles.

### BACKGROUND

Repositioning the papillary muscles within the ventricles of the heart during atrioventrical valve repair surgery improves outcomes. The displacement of the papillary muscles, due to ischemia, heart failure, or other causes of ventricular reshaping, results in tethering of the valve leaflets which interferes with their normal functioning. Repairs that focus only on the valve annulus often result in recurrence of regurgitation due to leaflet tethering. Papillary muscle approximation has been shown to improve the function of the mitral valve and the left ventricle in patients suffering from heart failure and mitral regurgitation.

Current methods of papillary muscle repositioning are typically performed during an open heart surgery. Therefore, there is a need for a less invasive device and method of repositioning and appositioning papillary muscles.

### SUMMARY

Presently disclosed embodiments recognize that a need exists for improved devices and methods for transcatheter navigation to a cavity within the body, and adjustment within the cavity. The embodiments of the present disclosure include devices and methods of transcatheter navigation to a cavity within the body, for example to a ventricle of a heart. Advantageously, the exemplary embodiments provide devices and methods of transcatheterly navigating a band to a ventricle of a heart such that the band can be adjusted to apposition the papillary muscles in the ventricle.

For example, a papillary muscle band which can be implanted around the base of the papillary muscles in a ventricle of a heart via a catheter can allow transcatheter papillary muscle approximation. The band used in such transcatheter procedures may have specific requirements that may not be required for conventional methods of papillary muscle repositioning during an open heart surgery. By way of example, a primary requirement may be that the band be configured to be inserted through a catheter and then formed into a band around the base of the papillary muscles with an adjustable circumference. Because the band may need to be transcatheterly inserted and formed into a band around the papillary muscles, the band may also need a means for remotely adjusting the circumference of the band, and thereby the extent of papillary muscle approximation, in a reversible manner, a locking mechanism that may be actuated via the catheter, and/or an ability to disconnect the band from the insertion catheter. The embodiments of the present disclosure provide a papillary muscle band including appropriate features to allow the band to be implanted around the papillary muscles via a catheter. Various embodiments of the disclosure may include one or more of the following aspects.

Consistent with an embodiment of the present disclosure, a device for repositioning papillary muscles is provided, the device comprising a band configured to at least partially surround a cluster of papillary muscles, and a winch coupled to a location proximate a first end of the band. The cluster of papillary muscles may have an outer peripheral boundary, and the band may include the first end and a second end. The winch may be configured to adjust a length of the band to apposition the papillary muscles in the cluster.

In some embodiments, the device may further comprise a pullwire, and the winch may be configured to pull the pullwire to adjust the length of the band. In other embodiments, the pullwire may be coupled to a location proximate the second end of the band. In some embodiments, the band may be configured to form a closed loop around the cluster of papillary muscles, and the winch may be configured to adjust the length of the band to change a circumference of the closed loop. The winch may be configured to pull on the first end and the second end of the band to decrease the circumference of the closed loop. In other embodiments, the winch may be configured to pull on strings coupled to the first end and the second end of the band to decrease the circumference of the closed loop.

In some embodiments, the band may be configured to form an open C-shaped partial loop around the cluster of papillary muscles, and an adjustment of the winch may be configured to change a shape of the band to apposition the papillary muscles in the cluster. In some embodiments, the band may be configured to bend inward when the winch is adjusted, thereby appositioning the papillary muscles in the cluster. In other embodiments, the band may include at least one of a flexible polymer, a cut metal tube, or a series of interconnected segments configured to rotate relative to each other.

According to another embodiment of the present disclosure, a device for repositioning papillary muscles is provided, the device comprising a band configured to form a loop around a cluster of papillary muscles. The cluster may have an outer peripheral boundary defined by the loop, and the band may include a first end and a second end. The first end and the second end of the band may be configured to be twisted together to adjust a circumference of the loop formed by the band and to lock the band at a preferred circumference.

According to yet another embodiment of the present disclosure, a device for repositioning papillary muscles is provided, the device comprising a band configured to be inserted into a ventricle of a heart to form a loop around a cluster of papillary muscles. During insertion of the band into the ventricle of the heart, the band may be configured to have a spiral shape to make at least one full revolution around the cluster of papillary muscles.

In some embodiments, the device may further comprise a shape memory alloy configured to modify the shape of the band after insertion of the band into the ventricle of the heart such that the band may be configured to apposition the papillary muscles in the cluster. In other embodiments, the shape memory alloy may be configured to be heated after insertion of the band into the ventricle of the heart to change the shape of the band. Additionally or alternatively, the shape memory alloy may be configured to be cooled during insertion of the band into the ventricle of the heart to maintain the shape of the band, and then the shape memory alloy mayheat up to body temperature to change the shape of the band.

Consistent with yet another embodiment of the present disclosure, a device for repositioning papillary muscles is provided, the device comprising a band configured to form an open C-shaped partial loop around a cluster of papillary muscles. The cluster may have an outer peripheral boundary partially defined by the partial loop. The band may be configured to be adjusted to change a shape of the band and apposition the papillary muscles in the cluster.

In some embodiments, the device may further comprise a pullwire coupled to an end of the band, and the pullwire may be configured to be pulled to adjust the band. In some embodiments, the device may further comprise a pullwire pulling mechanism. The pullwire pulling mechanism may be configured to be actuated by a rotation of a component of the pullwire pulling mechanism. In other embodiments, the pullwire pulling mechanism may include a screw. In other embodiments, the device may further comprise a pullwire ratchet mechanism configured to allow the pullwire to pass through in only one direction.

In some embodiments, the band may be configured to lock into a bent shape upon being adjusted such that when an adjustment force is released, the band may be configured to maintain the bent shape. In some embodiments, the band may include a metal tube cut into a pattern, and the pattern may form latches configured to lock adjacent segments of the metal tube when the band is bent. In some embodiments, the band may be configured to be locked into a geometric configuration at any time while the band is being adjusted. The band may be configured to be locked into the geometric configuration by filling the band with a material that hardens after insertion into the band.

Consistent with another embodiment of the present disclosure, a device for repositioning papillary muscles is provided, the device comprising a band configured to at least partially surround a cluster of papillary muscles. The cluster may have an outer peripheral boundary. The band may include an inflatable sealed tube, and the sealed tube may be configured to form a predetermined shape when inflated. In some embodiments, the sealed tube may be configured to be inflated after being passed around the papillary muscles, and the sealed tube may be configured to form the predetermined shape after inflation to apposition the papillary muscles. In other embodiments, the band may be configured to be locked when the sealed tube is inflated to maintain the appositioning of the papillary muscles. In some embodiments, the band may be configured to be inflated with a material that hardens within the band, thereby maintaining an inflated shape to apposition the papillary muscles.

Additional objects and advantages of the embodiments will be set forth in part in the description that follows, and in part will be obvious from the description or may be learned by practice of the embodiments. The objects and advantages of the embodiments will be realized and attained by means of the elements and combinations particularly pointed out in the appended claims.

It is to be understood that both the foregoing general description and the following detailed description are exemplary and explanatory only and are not restrictive of the claims.

### BRIEF DESCRIPTION OF THE DRAWINGS

Some features of disclosed embodiments are set forth with particularity in the claims that follow. Additional details of the features and advantages of the disclosed embodiments will be obtained by reference to the following detailed description that sets forth illustrative examples, in which the disclosed principles are utilized, and the accompanying drawings of which:

FIG. 1A illustrates an exemplary device for repositioning papillary muscles, consistent with an embodiment of the present disclosure;

FIG. 1B illustrates another view of the exemplary device for repositioning papillary muscles of FIG. 1A, consistent with an embodiment of the present disclosure;

FIG. 2 illustrates another exemplary device for repositioning papillary muscles, consistent with an embodiment of the present disclosure;

FIG. 3 illustrates another exemplary device for repositioning papillary muscles, consistent with an embodiment of the present disclosure;

FIG. 4 illustrates another exemplary device for repositioning papillary muscles, consistent with an embodiment of the present disclosure;

FIG. 5A illustrates another exemplary device for repositioning papillary muscles in a first mode, consistent with an embodiment of the present disclosure;

FIG. 5B illustrates the exemplary device for repositioning papillary muscles of FIG. 5A in a second mode, consistent with an embodiment of the present disclosure;

FIG. 6 illustrates another exemplary device for repositioning papillary muscles, consistent with an embodiment of the present disclosure;

FIG. 7A illustrates an exemplary device for repositioning papillary muscles, the device having a band placed around the papillary muscles in a heart, consistent with an embodiment of the present disclosure;

FIG. 7B illustrates the exemplary device for repositioning papillary muscles of FIG. 7A after the band has been adjusted to apposition the papillary muscles, consistent with an embodiment of the present disclosure;

FIG. 7C illustrates the exemplary device for repositioning papillary muscles of FIGS. 7A and 7B after the band has been inflated, consistent with an embodiment of the present disclosure;

FIG. 8A illustrates an exemplary torque cable rotating an exemplary winch in a first direction, consistent with an embodiment of the present disclosure;

FIG. 8B illustrates the exemplary torque cable disconnecting from the exemplary winch of FIG. 8A, consistent with an embodiment of the present disclosure;

FIG. 9 illustrates an exemplary cut pattern on a wall of a band, consistent with an embodiment of the present disclosure;

FIG. 10 illustrates an exemplary shape locking mechanism, consistent with an embodiment of the present disclosure;

FIG. 11A illustrates an exemplary ratchet, consistent with an embodiment of the present disclosure;

FIG. 11B illustrates another exemplary ratchet, consistent with an embodiment of the present disclosure;

FIG. 12 illustrates an exemplary method of twisting the ends of a band, consistent with an embodiment of the present disclosure;

FIG. 13 illustrates an exemplary band composed of a non-elastic balloon, consistent with an embodiment of the present disclosure;

FIG. 14A illustrates an exemplary ball valve near an opening of a non-elastic balloon, consistent with an embodiment of the present disclosure;

FIG. 14B illustrates an exemplary flap valve near an opening of a non-elastic balloon, consistent with an embodiment of the present disclosure;

FIG. 14C illustrates an exemplary adhesive sealant near an opening of a non-elastic balloon, consistent with an embodiment of the present disclosure;

FIG. 15 illustrates a 2-component adhesive material in an exemplary band, consistent with an embodiment of the present disclosure;

FIG. 16A illustrates a first component adhesive material in an exemplary band, consistent with an embodiment of the present disclosure; and

FIG. 16B illustrates a second component adhesive material inserted inside the exemplary band of FIG. 16A, consistent with an embodiment of the present disclosure.

### DETAILED DESCRIPTION OF EMBODIMENTS

The present disclosure relates to methods and devices for transcatheter insertion and navigation to a cavity within a body. While the present disclosure provides examples of transcatheterly navigating to a ventricle of a heart via a device for repositioning the plurality of papillary muscles, it should be noted that aspects of the disclosure in their broadest sense, are not limited to devices for manipulation around a papillary muscle between a trabeculae. Rather, it is contemplated that the forgoing principles may be applied to other devices for transcatheter navigation to any cavity within a body.

In addition, the term “band” refers generally to any element that is capable of extending, encircling, and/or looping around an object, such as a muscle in the body. For example, a band may be a wire, a filament, a strap, a tube, an inflatable tube, a sling, or any elongated tool with or without a channel or a cavity therein. Moreover, the term “pullwire” refers generally to any element that is capable of extending through a band. For example, a pullwire may be a string, a wire, astrand , and/or a flexible tube. Furthermore, the term “apposition” refers generally to any type of movement to bring objects closer to each other. For example, “appositioning” may be bringing objects closer to each other or bringing objects in contact with each other. The terms “apposition” and “approximate” may be used interchangeably herein.

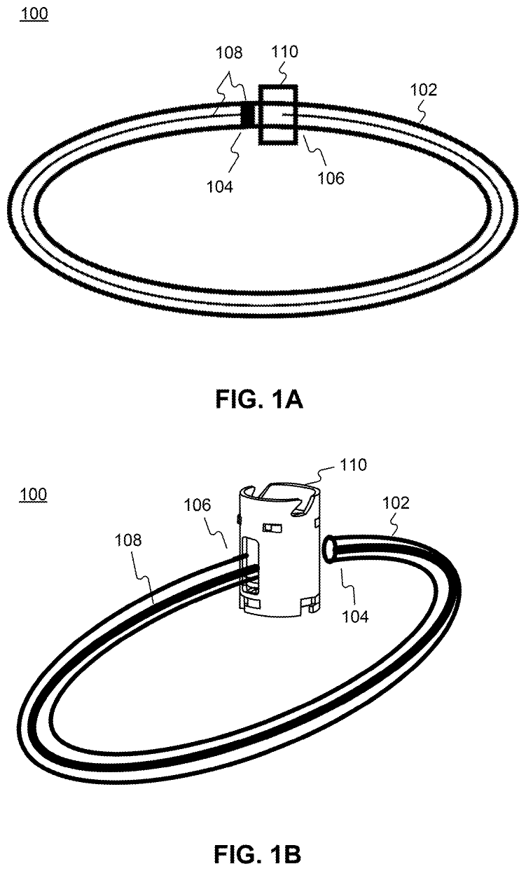

Referring to FIGS. 1A and 1B, an exemplary device 100 for transcatheter navigation to a cavity, such as a heart, within a body, consistent with the present disclosure, may include a band 102. Band 102 may include a first end 106 and a second end 104. Band 102 may also include a winch 110 coupled to a position proximate the first end 106 of band 102. In some embodiments, winch 110 may be coupled to the first end 106 of band 102. Additionally or alternatively, band 102 may include a pullwire 108 coupled to a position proximate the second end 104 of band 102. In some embodiments, pullwire 108 may be coupled to the second end 104 of band 102. A winch may refer generally to any device that is capable of winding a string, a wire, a band, a pullwire, a sling, and/or a flexible tube around it or within it. In addition, as discussed above, a pullwire may refer generally to any element that is capable of extending through a band. For example, a pullwire may be a string, a wire, astrand , and/or a flexible tube.

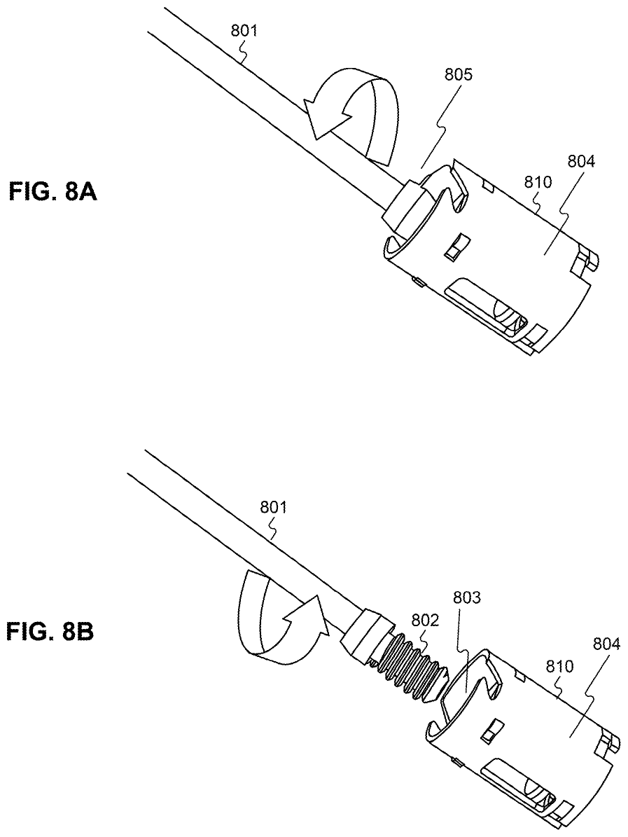

In some embodiments, as shown in FIGS. 8A and 8B, a winch 810 may include an outer shell 804 and an inner rotating shaft 803. The outer shell 804 may have an opening 805 through which passes a band, such as band 102 of FIG. 1, string, or wire to be pulled. The band, string, or wire may be connected to the inner rotating shaft 803 and become wound around the inner rotating shaft 803 when the inner rotating shaft 803 rotates. The inner rotating shaft 803 may be rotated by rotation of an actuator on a handle of a catheter connected to the inner rotating shaft 803 via a torque cable 801 running through the catheter. The torque cable 801 may be removably attached to the inner rotating shaft 803 of the winch 810, such that it can be disconnected after tightening the band, such as band 102. Additionally or alternatively, the torque cable 801 may have a screw 802 at the end that screws into the inner rotating shaft 803 of the winch 801, such that when rotated in one direction, the rotation of the torque cable 801 rotates the inner rotating shaft 803 (as shown in FIG. 8A), and when rotated in the other direction, the torque cable 801 unscrews from the inner rotating shaft 803 and disconnects from the winch 810 (as shown in FIG. 8B). The inner rotating shaft 803 of the winch 810 may optionally include a ratchet mechanism such that it can rotate in only one direction. The ratchet mechanism would allow the winch 810 to tighten the band, such as band 102 of FIG. 1, but would not allow the band to loosen.

In some embodiments, band 102 may be shaped, sized, and configured to be inserted into a cavity within a body. For example, band 102 may be shaped, sized, and configured to be transcatheterly inserted into a heart of a body and placed around a cluster of papillary muscles in a ventricle of the heart. The cluster of papillary muscles may have an outer peripheral boundary. Additionally or alternatively, band 102 may be configured to be passed around and navigated through the spaces between trabeculae. In some embodiments, band 102 may be configured to be placed around the base of the papillary muscles in the ventricle of the heart.

In some embodiments, after placing band 102 around multiple papillary muscles, band 102 may be configured to form a closed loop around the papillary muscles, thereby encircling the papillary muscles. Additionally or alternatively, band 102 may be configured to partially surround multiple papillary muscles to form an open C-shaped partial loop around the papillary muscles. In other embodiments, after placing band 102 around multiple papillary muscles the first end 106 and the second end 104 of band 102 may be configured to contact each other without being coupled to each other.

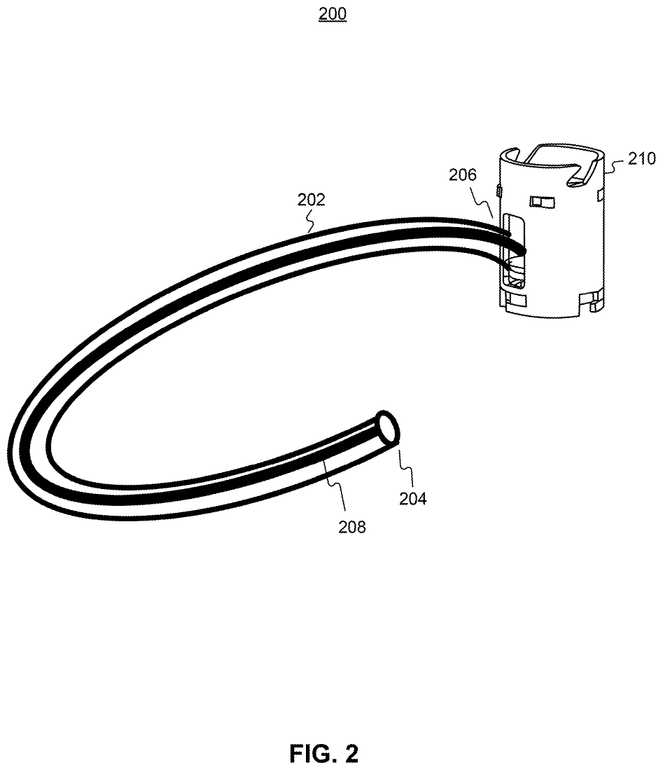

Band 102 may form a closed loop (for example, as illustrated in FIGS. 1A and 1B) or an open C-shaped partial loop (for example, as illustrated in FIG. 2) around the papillary muscles by an actuation of winch 110. By way of example, winch 110 may be actuated to adjust band 102 by winding band 102 and/or pullwire 108 around winch 110. As such, upon actuation of winch 110, band 102 may be adjusted such that a circumference of the closed loop or the open C-shaped partial loop around the papillary muscles decreases, thereby causing appositioning of the papillary muscles. For example, upon actuation of winch 110, winch 110 may be configured to wind band 102 and/or pullwire 108 to reduce the circumference of the closed loop or to bend the open C-shaped partial loop inward. By reducing the circumference of the closed loop or bending the open C-shaped partial loop inward, band 102 may be configured to bring the papillary muscles closer to each other and apposition the papillary muscles. In some embodiments, winch 110 may include a mechanism to avoid band 102 and/or pullwire 108 from loosening after band 102 and/or pullwire 108 has been tightened. For example, winch 110 may include a ratchet or a lock to avoid band 102 and/or pullwire 108 from loosening after band 102 and/or pullwire 108 has been tightened.

In other embodiments, the closed loop or the open C-shaped partial loop formed around the papillary muscles by band 102 may be adjusted by an actuation of pullwire 108. By way of example, upon actuation of pullwire 108, pullwire 108 may be configured to adjust band 102 and adjust a circumference of the closed loop or the open C-shaped partial loop around the papillary muscles, thereby causing appositioning of the papillary muscles. For example, upon actuation of pullwire 108, pullwire 108 may be configured to pull band 102 to reduce the circumference of the closed loop or to bend the open C-shaped partial loop inward. By reducing the circumference of the closed loop or bending the open C-shaped partial loop inward, band 102 may be configured to bring the papillary muscles closer to each other and apposition the papillary muscles.

In some embodiments, band 102 may include a flexible tube with pullwire 108 running through the tube. Pullwire 108 may be coupled to the second end 104 of band 102. In addition, as illustrated in FIGS. 1A and 1B, winch 110 may be coupled to the first end 106 of band 102. Winch 110 may be configured to wind and pull pullwire 108 to adjust a length or shape of band 102 and/or adjust the closed loop or the open C-shaped partial loop formed by band 102 around the papillary muscles. For example, winch 110 may be configured to wind and pull pullwire 108 to reduce the circumference of the closed loop or to bend the open C-shaped partial loop inward. Although FIGS. 1A and 1B illustrate a loop formed by band 102, band 102 may be configured to form any geometric shape, for example around the papillary muscles. By way of example, band 102 may be configured to form a square, a triangle, a rectangle, a hexagon, or any other regular or irregular geometric shape around the papillary muscles.

In other embodiments, pullwire 108 may be configured to pass through both the first end 106 and the second end 104 of band 102. Additionally or alternatively, winch 110 may be coupled to the first end 106 and/or the second end 104 of band 102. Accordingly, winch 110 may be configured to pull on both ends of pullwire 108 to adjust a shape of band 102. For example, winch 110 may be configured to pull on both ends of pullwire 108 to reduce the circumference of the closed loop. Additionally or alternatively, instead of pulling on both ends of pullwire 108, winch 110 may be configured to pull directly on the first end 106 and the second end 104 of band 102 to adjust a shape and/or circumference of band 102 around the papillary muscles. Additionally or alternatively, winch 110 may be configured to pull on one or more strings and/or one or more wires coupled to the first end 106 and the second end 104 of band 102 to adjust a shape and/or circumference of band 102 around the papillary muscles.

Referring now to FIG. 2, an exemplary device 200 for transcatheter navigation to a cavity, such as a heart, within a body, consistent with the present disclosure, may include a band 202. Band 202 may include a first end 206 and a second end 204. Band 202 may also include a winch 210 coupled to a position proximate the first end 206 of band 202. In some embodiments, winch 210 may be coupled to the first end 206 of band 202. Additionally or alternatively, band 202 may include a pullwire 208 coupled to a position proximate the second end 204 of band 202. In some embodiments, pullwire 208 may be coupled to the second end 204 of band 202.

In some embodiments, band 202 may be constructed of a flexible tube that is configured to bend into an open C-shaped partial loop when tension is applied to pullwire 208 running through band 202. For example, upon actuation of pullwire 208, band 202 may be configured to bend into the open C-shaped partial loop as shown in FIG. 2. Additionally or alternatively, winch 210 may be configured to pull on pullwire 208 to induce band 102 to bend into the open C-shaped partial loop, thereby appositioning the papillary muscles. For example, upon actuation of winch 210, winch 210 may be configured to pull pullwire 208 and induce band 202 to bend into the open C-shaped partial loop. When band 202 is induced to bend into the open C-shaped partial loop, band 202 placed around the papillary muscles may be configured to bring the papillary muscles closer to each other, thereby appositioning the papillary muscles.

In some embodiments, band 202 may include a flexible tube made of a flexible polymer material, a cut metal tube, or a series of interconnected segments configured to rotate relative to each other. For example, band 202 may include an elongated tubular structure with a cut pattern in the wall of the tube. The cut pattern may form segments that are configured to be interconnected to each other or interlocked together to form band 202. The cut segments may allow band 202 to have multiple mechanical modes and/or configurations, such that in one configuration, band 202 may be flexible in multiple directions, and in another configuration, band 202 may be rigid in a pre-defined shape.

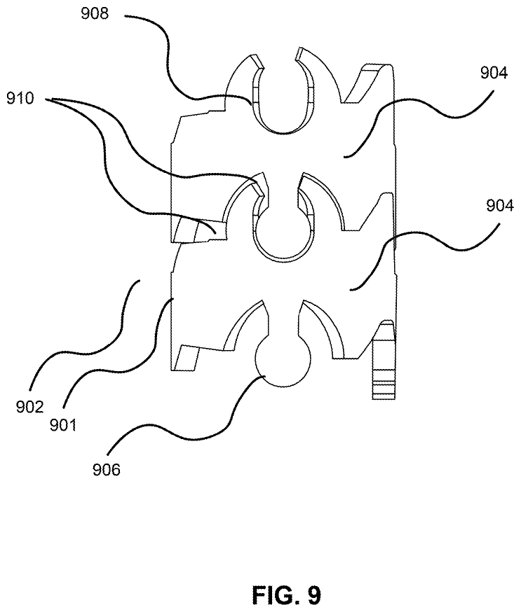

For example, as shown in FIG. 9, band 902, which may be implemented as band 202 of FIG. 2, may include an elongated tubular structure 901 with a cut pattern in the wall of the structure 901 to form a plurality of segments 904. Each segment 904 may include a male joint 906 and a female joint 908. By increasing the freedom of motion in female joints 908 of segments 904, male joints 906 may be able to move slightly out of the pockets created by female joints 908. Therefore, because male joints 906 may have more freedom of motion in female joints 908, segments 904 may be able to rotate slightly relative to an adjacent segment 904 in directions both perpendicular to and opposite the predefined rotation that segments 904 are designed to take in a rigid configuration. That is, male joints 906 may be able to move in all directions within female joints 908. The cut pattern on segments 904 may also vary in order to allow segments 904 to rotate in one or more directions. For example, the cut pattern may provide gaps 910 on two different sides of the elongated tubular structure 901 of band 902 such that segments 904 may be able to rotate relative to each other in two directions. Additionally or alternatively, the cut pattern may provide three or more gaps on various sides of the elongated tubular structure 901 of band 902 in order to form a desired rigid, pre-defined shape.

While the cut pattern may provide flexibility at each segment 904 to the entire region of the elongated tubular structure 901 of band 902 containing the cut pattern, band 902 may become rigid when actuated by a strand, such as pullwire 208 of FIG. 2. When actuated by pullwire 208, for example, pullwire 208 may push male joints 906 into female joints 908 of adjacent segments 904, and thus, remove the freedom of motion in female joints 908 and force segments 904 into their rigid, pre-defined configuration.

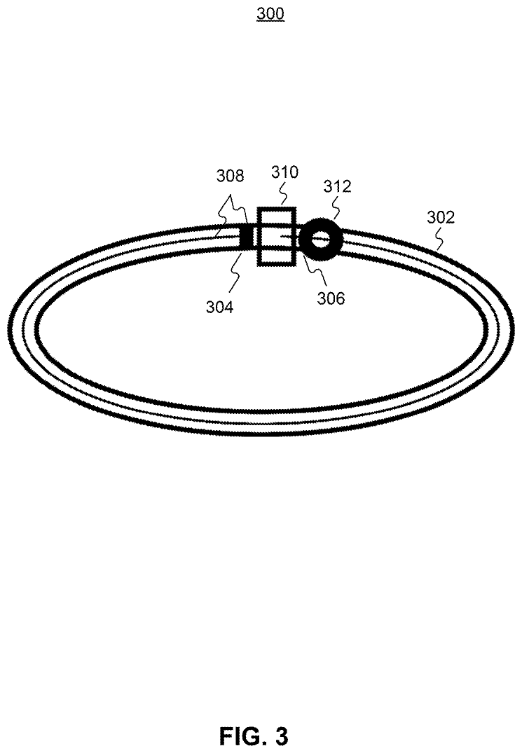

Referring now to FIG. 3, an exemplary device 300 for transcatheter navigation to a cavity, such as a heart, within a body, consistent with the present disclosure, may include a band 302. Band 302 may include a first end 306 and a second end 304. Band 302 may also include a winch 310 coupled to a position proximate the first end 306 of band 302. In some embodiments, winch 310 may be coupled to the first end 306 of band 302. Additionally or alternatively, band 302 may include a pullwire 308 coupled to a position proximate the second end 304 of band 302. In some embodiments, pullwire 308 may be coupled to the second end 304 of band 302.

As shown in FIG. 3, band 302 may also include a pullwire ratchet mechanism including a ratchet 312. As discussed above, band 302 may form a closed loop or an open C-shaped partial loop around the papillary muscles by an actuation of winch 310. By way of example, winch 310 may be actuated to adjust band 302 by winding band 302 and/or pullwire 308 around winch 310. As such, upon actuation of winch 310, band 302 may be adjusted such that a circumference of the closed loop or the open C-shaped partial loop around the papillary muscles decreases, thereby causing appositioning of the papillary muscles. For example, upon actuation of winch 310, winch 310 may be configured to wind band 302 and/or pullwire 308 to reduce the circumference of the closed loop or to bend the open C-shaped partial loop inward. By reducing the circumference of the closed loop or bending the open C-shaped partial loop inward, band 302 may be configured to bring the papillary muscles closer to each other and apposition the papillary muscles. After shortening pullwire 308, for example, ratchet 312 may be configured to lock pullwire 308 such that pullwire 308 cannot be loosened. For example, ratchet 312 may be configured to allow pullwire 308 to pass through in only one direction. Accordingly, ratchet 312 may be configured to lock pullwire 308 to maintain the shape of band 302 and prevent band 302 from loosening its loop around the papillary muscles.

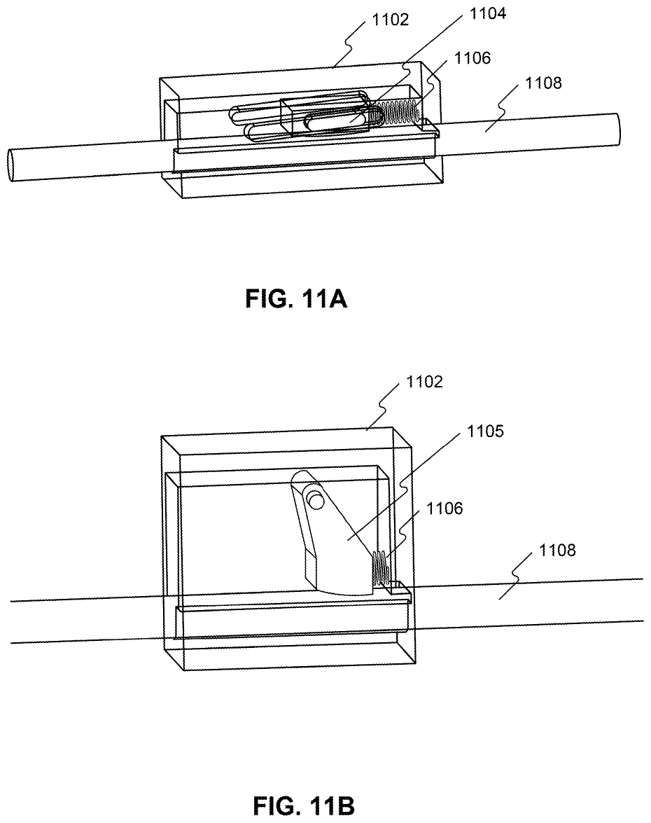

In some embodiments, as illustrated in FIGS. 11A and 11B, ratchet 312 may be a ratchet 1102 configured to apply frictional force on pullwire 1108, which may be implemented as pullwire 308, when pullwire 1108 is pulled through ratchet 1102 in one direction, and not to apply frictional force when pullwire 1108 is pulled in the other direction. The directionally selective frictional force may be induced by a sliding component 1104 in FIG. 11A that is tightened against pullwire 1108 by the movement of pullwire 1108 in one direction, or by a hinged component 1105 in FIG. 11B that is tightened against pullwire 1108 by the movement of pullwire 1108 in one direction, or by any other directionally selective ratchet mechanism known in the art. A spring 1106 may keep the sliding friction component 1104 in FIG. 11A or the hinged component 1105 in FIG. 11B in contact with the pullwire 1108, such that if pullwire 1108 is pulled to the left, spring 1106 may pull the sliding friction component 1104 in FIG. 11A or the hinged component 1105 in FIG. 11B tighter against itself, thereby locking pullwire 1108 in place. On the other hand, if pullwire 1108 is pulled to the right, the movement may loosen the sliding friction component 1104 in FIG. 11A or the hinged component 1105 in FIG. 11B, thereby allowing pullwire 1108 to move with little resistance. In some embodiments, the effect of the frictional force of the ratchet mechanism may be enhanced by the properties of pullwire 1108. For example, pullwire 1108 may have a rough or grooved outer surface in order to enhance the effectiveness of ratchet 1102. Additionally or alternatively, pullwire 1108 may be made of a twisted or braided rope or cable or of a polymer material that can be deformed by the force of ratchet 1102 in order to enhance the effectiveness of ratchet 1102. In other embodiments, the sliding friction component 1104 in FIG. 11A or the hinged component 1105 in FIG. 11B and/or an inner surface of ratchet 1102 that pullwire 1108 contacts can be rough or grooved to enhance the friction between pullwire 1108 and the sliding friction component 1104 in FIG. 11A or the hinged component 1105 in FIG. 11B and/or the inner surface of ratchet 1102, thereby improving the effectiveness of locking pullwire 1108.

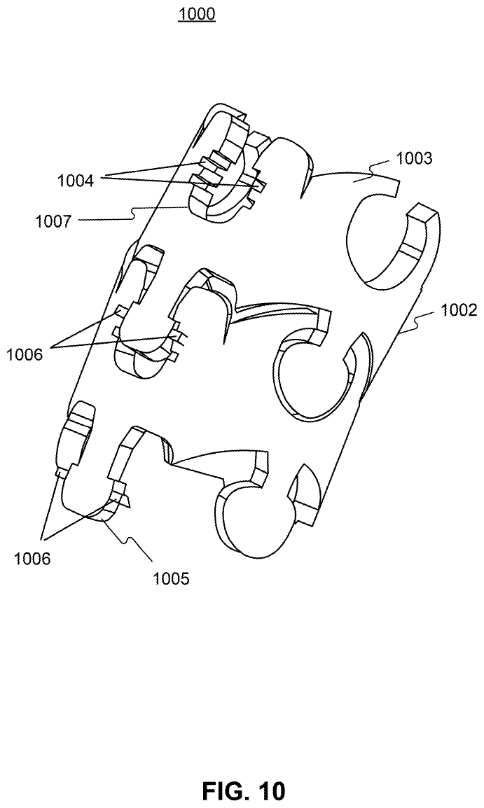

In other embodiments, device 300 may include a separate shape-locking mechanism that is configured to prevent band 302 from bending back into its original shape when tension in pullwire 308 is released. For example, FIG. 10 illustrates an exemplary shape locking mechanism 1000 according to some embodiments of the present disclosure. Shape locking mechanism 1000 may include latches 1006 formed on a male joint 1005 of segments 1003 of band 1002. Band 1002 may be made of a cut metal tube. Latches 1006 formed in male joints 1005 may fit into grooves 1004 formed in female joints 1007 of the segments 1003 to lock adjacent segments 1003 relative to each other. In some embodiments, when one segment 1003 rotates relative to the adjacent segment 1003, the segment 1003 may become locked when latches 1006 fit into grooves 1004, and the segment 1003 may not rotate back, thereby locking band 1002 into its bent configuration.

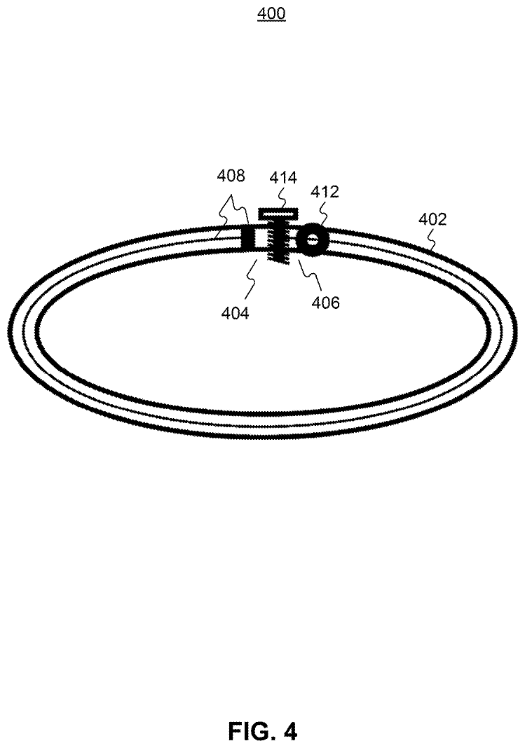

FIG. 4 illustrates another exemplary device 400 for transcatheter navigation to a cavity, such as a heart, within a body, consistent with the present disclosure. Device 400 may include a band 402. Band 402 may include a first end 406 and a second end 404. Band 402 may also include a pullwire 408 coupled to a position proximate the second end 404 of band 402. In some embodiments, pullwire 408 may be coupled to the second end 404 of band 402. In addition, band 402 may include a ratchet 412 and a pullwire pulling mechanism including a screw 414.

As discussed above in reference to FIG. 3, ratchet 412 may be configured to lock pullwire 408 such that pullwire 408 cannot be loosened. For example, ratchet 412 may be configured to allow pullwire 408 to move through ratchet 412 in only one direction. Accordingly, ratchet 412 may be configured to lock pullwire 408 to maintain the shape of band 402 around the papillary muscles and prevent band 402 from loosening its loop around the papillary muscles. In other embodiments, device 400 may include a separate shape-locking mechanism that is configured to prevent band 402 from bending back into its original shape when tension in pullwire 408 is released.

Additionally or alternatively, band 402 may include a pullwire pulling mechanism including a screw 414 that is configured to pull on and shorten pullwire 408. For example, the pullwire pulling mechanism may be actuated by a rotation of screw 414. Screw 414 may be configured to rotate to actuate the pullwire pulling mechanism and wind pullwire 408 around screw 414 to thereby pull and shorten pullwire 408. By shortening pullwire 408, screw 414 may be configured to reduce a circumference of a closed loop formed by band 402 around a plurality of papillary muscles or bend an open C-shaped partial loop formed by band 402 around the papillary muscles inward. By reducing the circumference of the closed loop or bending the open C-shaped partial loop inward, band 402 may be configured to bring the papillary muscles closer to each other and apposition the papillary muscles. For example, screw 414 may be the same as screw 802 of FIGS. 8A and 8B. As discussed above, screw 802 may be rotated by rotation of an actuator on a handle of a catheter connected to screw 802 via a torque cable 801 running through the catheter. The torque cable 801 may be removably attached to screw 802, such that torque cable 801 can be disconnected after tightening the band, such as band 402. Additionally or alternatively, screw 802 may comprise a threaded tip at the end of the torque cable 801 that screws into screw 414 which is configured to tighten the band, such as band 402. When rotated in one direction, the rotation of the torque cable 801 may be configured to rotate screw 414 and tighten the band, such as band 402 (as shown in FIG. 8A). When rotated in the other direction, the torque cable 801 may be configured to unscrew from the screw 414 and disconnect from the band, such as band 402 (as shown in FIG. 8B).

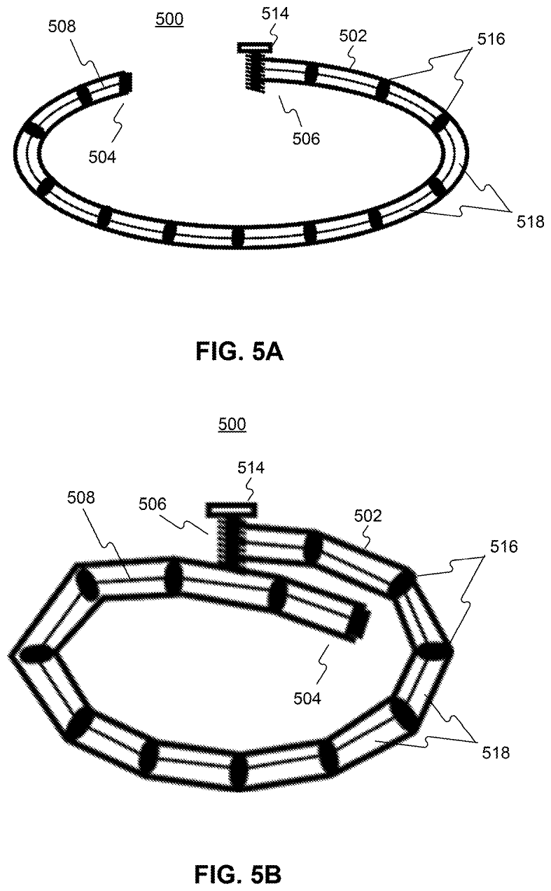

FIGS. 5A and 5B illustrate another exemplary device 500 for transcatheter navigation to a cavity, such as a heart, within a body, consistent with the present disclosure. Device 500 may include a band 502. Band 502 may include a first end 506 and a second end 504. Band 502 may also include a pullwire 508 coupled to a position proximate the second end 504 of band 502. In some embodiments, pullwire 508 may be coupled to the second end 504 of band 502. In addition, band 502 may include a screw 514 coupled to a position proximate the first end 506 of band 502. In some embodiments, screw 514 may be coupled to the first end 506 of band 502.

As discussed above, band 502 may be configured to form a closed loop or an open C-shaped partial loop around a plurality of papillary muscles in the ventricle of the heart. Additionally or alternatively, screw 514 may be configured to rotate to wind pullwire 508 around screw 514 to thereby shorten pullwire 508. By shortening pullwire 508, screw 514 may be configured to reduce a circumference of the closed loop formed by band 502 around a plurality of papillary muscles or bend the open C-shaped partial loop formed by band 502 around the papillary muscles inward. By reducing the circumference of the closed loop or bending the open C-shaped partial loop inward, band 502 may be configured to bring the papillary muscles closer to each other and apposition the papillary muscles.

In some embodiments, band 502 may further include an elongated tubular structure with cuts in the wall of the tube to form a plurality of segments 518 that are configured to be interconnected to each other or interlocked together to form band 502. For example, the plurality of segments 518 may be interconnected to each other or interlocked together by a plurality of latches, such as locking hinges 516, disposed therebetween. Locking hinges 516 may be configured to lock band 502 into a pre-defined geometric configuration at any time while band 502 is being adjusted. In some embodiments, band 502 may be formed of a cut metal hypotube and the cut pattern in the wall of the tube may form the locking hinges 516 that are configured to lock adjacent segments 518 when segments 518 are bent. As discussed below, locking hinges 516 may maintain the shape of band 502 by preventing segments 518 from returning to their original configurations. In some embodiments, locking hinges 516 may include latches, such as latches 1006 of FIG. 10, configured to fit into grooves, such as grooves 1004 of FIG. 10, on adjacent segments.

Segments 518 and locking hinges 516 may allow band 502 to have multiple mechanical modes and/or configurations such that in a first configuration, band 502 may be loosened and flexible in multiple directions (for example, as shown in FIG. 5A), and in a second configuration, band 502 may be tightened by pullwire 508 and rigid in a pre-defined shape (for example, as shown in FIG. 5B). For example, before screw 514 pulls on pullwire 508 to tighten band 502, band 502 may be loose and flexible as shown in FIG. 5A. Upon actuation of the pullwire pulling mechanism by a rotation of screw 514, screw 514 may pull on pullwire 508 to tighten band 502, thereby inducing band 502 to take on a rigid, bent shape as shown in FIG. 5B. When band 502 is tightened, the plurality of segments 518 may move and/or rotate relative to each other to form the rigid, bent shape as shown in FIG. 5B. After band 502 is tightened, locking hinges 516 may be configured to lock the plurality of segments 518 relative to each other such that band 502 is locked into the rigid, bent shape as shown in FIG. 5B after tightening without being loosened. Accordingly, even when the adjustment force by pullwire 508 is released, band 502 may be configured to maintain the rigid, bent shape as shown in FIG. 5B. In some embodiments, the pre-defined shape of band 502 when tightened may be defined by the shape of segments 518.

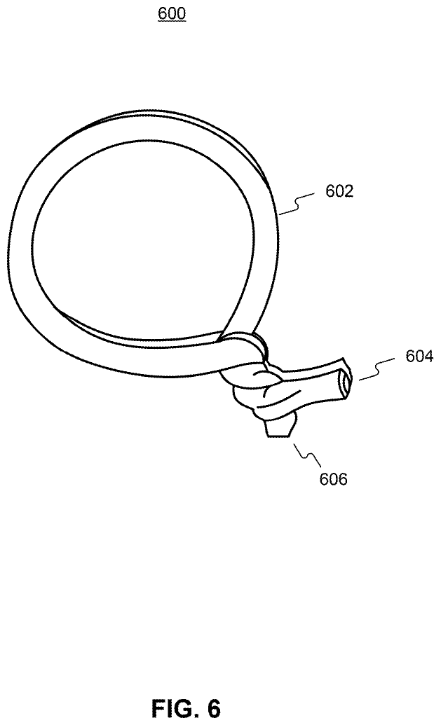

Referring now to FIG. 6, another exemplary device 600 for transcatheter navigation to a cavity, such as a heart, within a body, consistent with the present disclosure, may include a band 602. Band 602 may include a first end 606 and a second end 604. In some embodiments, band 602 may include a flexible tube made of a flexible polymer material, a cut metal tube, or a plurality of segments interconnected to each other. Band 602 may be configured to at least partially surround a plurality of papillary muscles so as to form a closed loop and/or an open C-shaped partial loop around the plurality of papillary muscles. In some embodiments, after band 602 is passed around the plurality of papillary muscles, a circumference of the closed loop formed by band 602 around the papillary muscles may be reduced by twisting the first end 606 and the second end 604 around each other, as shown in FIG. 6. Additionally or alternatively, the first end 606 and the second end 604 of band 602 may be made of a material with properties such that when the first end 606 and the second end 604 are twisted around each other, the first end 606 and the second end 604 are capable of maintaining its shape. By way of example, the first end 606 and the second end 604 are configured to stay twisted and become locked to each other in the twisted form. In some embodiments, the first end 606 and the second end 604 may be made of a soft metal material.

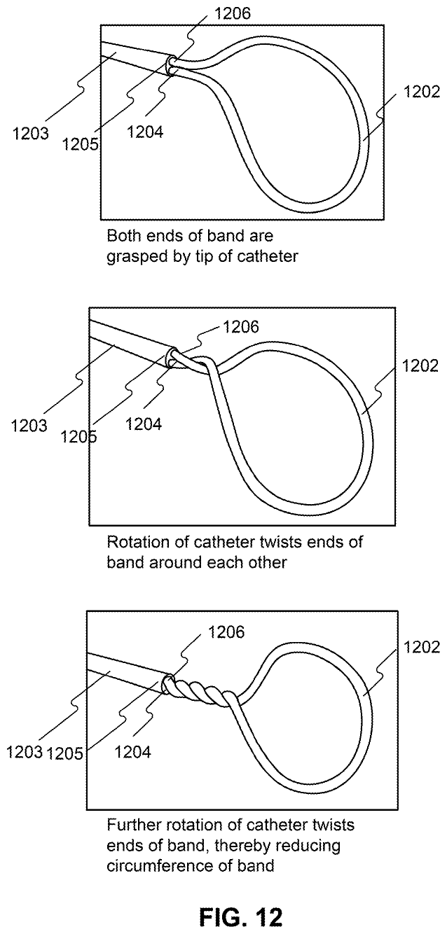

In some embodiments first end 606 and the second end 604 may be connected to a rotating component at a tip of a catheter which can be controllably rotated by rotating an actuator on the handle of the catheter. For example, as shown in FIG. 12, first end 1206 and second end 1204 of band 1202 may be connected to tip 1205 of catheter 1203. Catheter 1203 may be controllably rotated by rotating an actuator on a handle of catheter 1203. Optionally, the actuator on the handle may rotate a rotating component at tip 1205 of catheter 1203 via a torque cable, such as torque cable 801 of FIGS. 8A and 8B, running through the length of catheter 1203. In some embodiments, rotation of the rotating component at tip 1205 of catheter 1203 may twist first end 1206 and the second end 1204 around each other thereby reducing the circumference of band 1202.

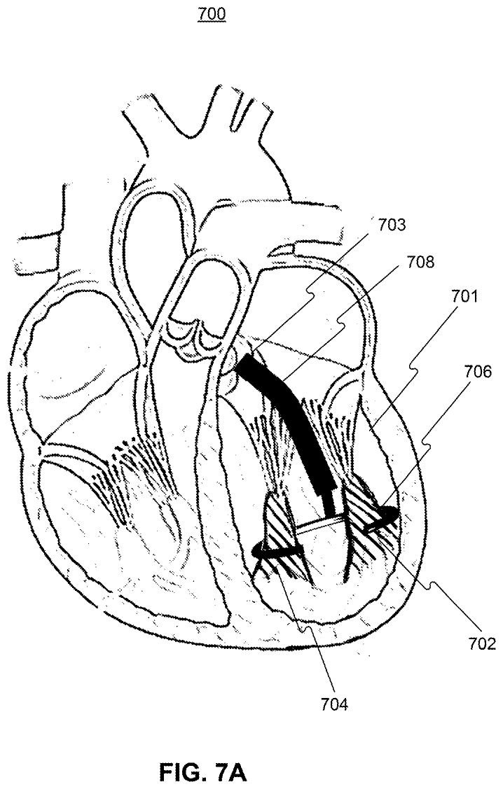

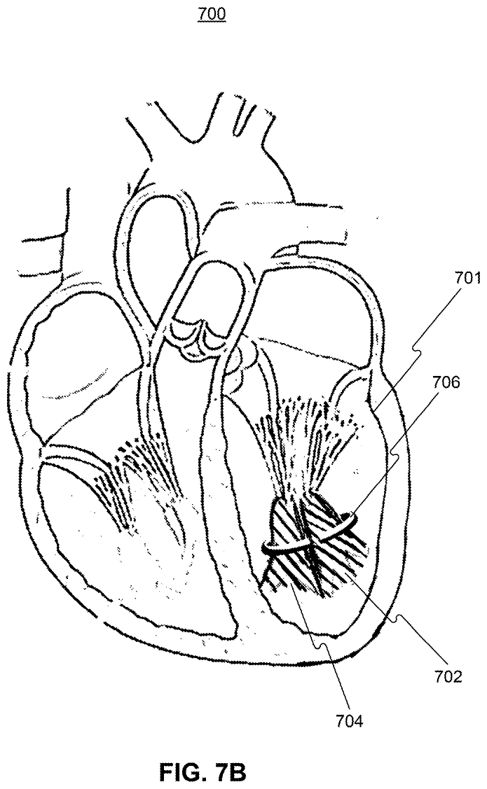

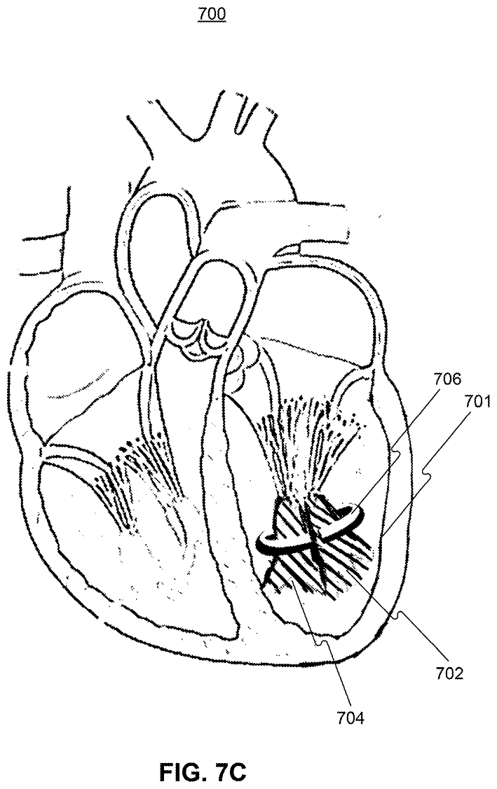

FIGS. 7A-7C illustrate an exemplary device 700 for transcatheter navigation to a dilated left ventricle 701 of a heart, consistent with the present disclosure. The term “transcatheter” or “transcatheterly” may include delivering via a catheter 708. Device 700 may include a band 706 configured to at least partially surround a cluster of papillary muscles 702, 704 to form a closed loop and/or an open C-shaped partial loop around the papillary muscles 702, 704.

FIG. 7A, for example, illustrates a single catheter 708 used to deliver band 706 through the aortic valve 703 to the left ventricle 701 in a heart. Band 706 may be releasably coupled to catheter 708. A transcatheter approach to the ventricle of the heart may be transthoracic, transarterial, transvenous, transseptal, transapical, or transatrial. All of these can be achieved using standard medical catheters and catheterization tools known in the art. They may also be performed using new tools for performing associated functions. As shown in FIGS. 7A-7C, band 706 may encircle, i.e., completely or partially surrounding, papillary muscles 702, 704. As discussed above, band 706 may be implemented as band 202 of FIG. 2, band 302 of FIG. 3, band 402 of FIG. 4, band 502 of FIGS. 5A-5B, and/or band 602 of FIG. 6. For example, band 706 may include a flexible tube made of a flexible polymer material, a cut metal tube, or a plurality of segments interconnected to each other. Catheter 708 may connect to band 706 at the first end of band 706, the second end of band 706, or anywhere along the length of band 706.

As shown in FIG. 7B, after transcatheter insertion of band 706 into the left ventricle 701 and after placing band 706 around papillary muscles 702, 704, band 706 may be tightened to form a closed loop around papillary muscles 702, 704. By way of example, band 706 may be adjusted and/or tightened by using a winch (such as winch 110 of FIGS. 1A-1B, winch 210 of FIG. 2, and/or winch 310 of FIG. 3), using a ratchet (such as ratchet 312 of FIG. 3 and/or ratchet 412 of FIG. 4), using a screw (such as screw 414 of FIG. 4 and/or screw 514 of FIGS. 5A-5B), using locking hinges (such as locking hinges 516 of FIGS. 5A-5B), twisting the ends of band 706 (as shown in FIG. 6), and/or any combination thereof. When band 706 is adjusted and/or tightened, the circumference of the closed loop around papillary muscles 702, 704 may be reduced to thereby apposition the papillary muscles 702, 704.

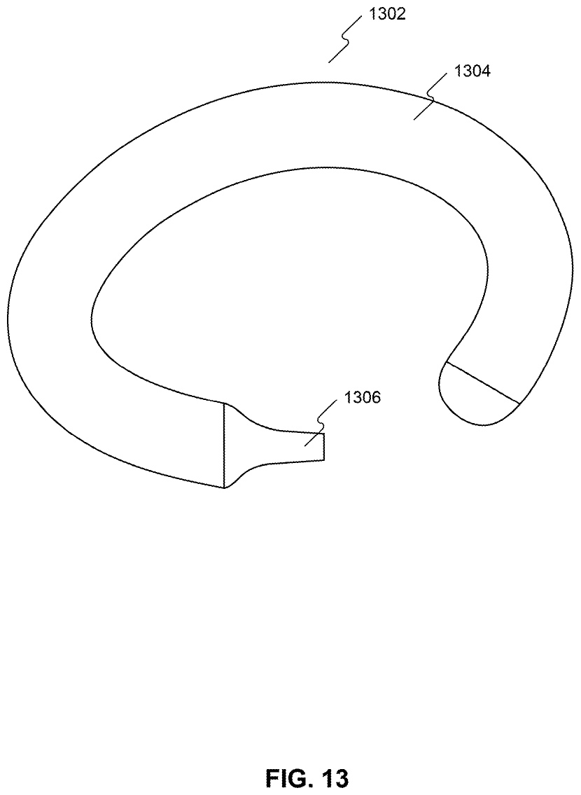

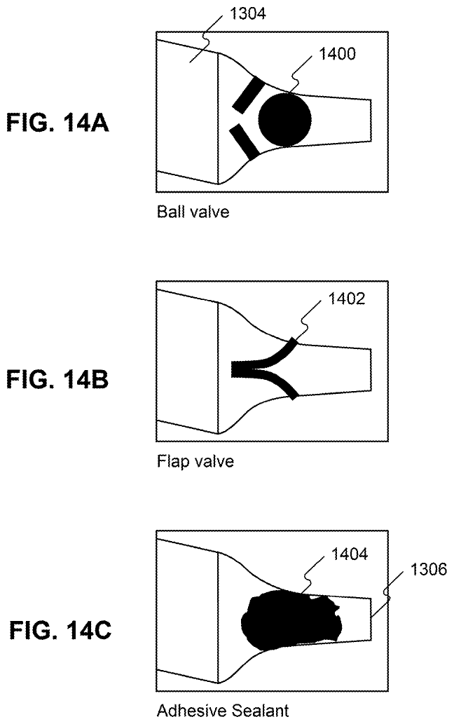

Additionally or alternatively, as shown in FIG. 7C, band 706 may be inflated to thereby cause band 706 to change to a pre-defined shape. Upon inflation of band 706, band 706 may change to the pre-defined shape to thereby apposition the papillary muscles 702, 704. By way of example, band 706 may be made of a shaped, sealed inflatable tube. In some embodiments, as shown in FIG. 13 for example, band 1302, which may be implemented as band 706, may be composed of a non-elastic balloon 1304. Non-elastic balloon 1304, when inflated forcefully, may take on the shape of the balloon as shown in FIG. 13. The composition and manufacturing of balloon 1304 may be well-known in the art of dilatation and angioplasty balloon catheter design. In some embodiments, balloon 1304 may include a one-way valve which may allow balloon 1304 to be inflated, but not to deflate. The one-way valve may be, for example, a ball valve 1400 (as shown in FIG. 14A), a flap valve 1402 (as shown in FIG. 14B), or any other type of one-way valve known in the art. Alternatively, anopening 1306 of balloon 1304 can be sealed once balloon 1304 is inflated by heating, twisting, inserting a plug, or inserting adhesive material. For example, as shown in FIG. 14C, opening 1306 of balloon 1304 may be sealed once inflated by an adhesive material 1404.

When the tube is inflated, band 706 may take on the pre-defined shape. Additionally or alternatively, the pre-defined shape may be an open C-shape, a circular shape, an oval shape, and/or any geometric shape or configuration that is configured to apposition the papillary muscles 702, 704. In some embodiments, once band 706 has been inserted and placed around papillary muscles 702, 704, band 706 may be inflated to apposition the papillary muscles 702, 704 and locked in the inflated configuration. Additionally or alternatively, band 706 may be inflated by inserting a polymer material inside the tubular structure of band 706. The polymer material may be configured to harden inside band 706 upon inflation to thereby lock band 706 into the pre-defined shape.

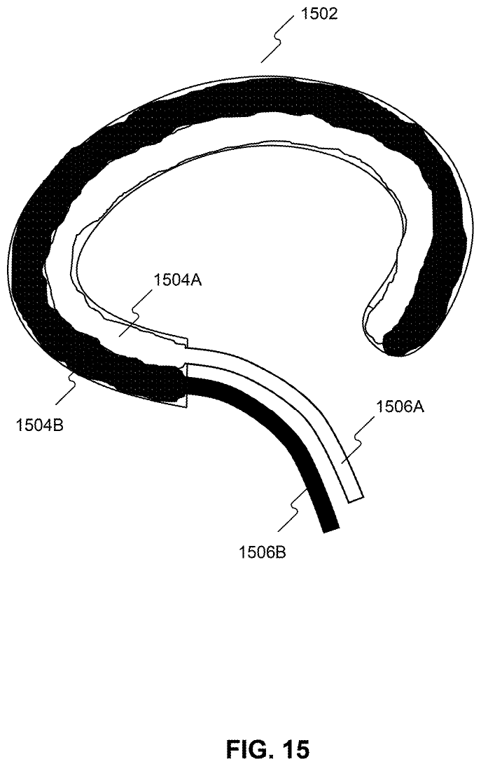

In some embodiments, the material may be a 2-component adhesive which hardens only when the two components come in contact with each other. For example, as shown in FIG. 15, band 1502, which may be implemented as band 706, may be filled with a first component adhesive 1504A through a first channel 1506A of a catheter and a second component adhesive 1504B through a second channel 1506B of the catheter. In some embodiments, the two adhesive components 1504A and 1504B may be inserted through two separate channels 1506A and 1506B in the catheter and contact each other only inside band 1502, thereby avoiding their hardening inside the channels 1506A and 1506B of the catheter.

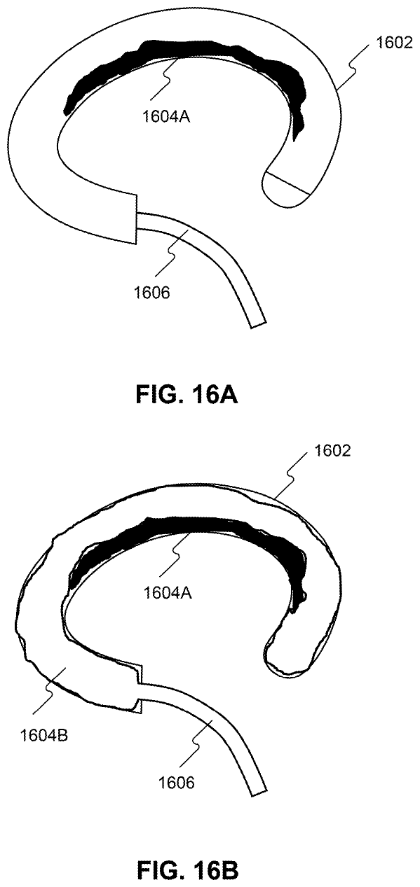

Additionally or alternatively, one adhesive component may be pre-existing inside the band, such as band 706, or coated onto the inside of the walls of the band or incorporated into the material of which the band is composed. For example, as shown in FIG. 16A, a first adhesive component 1604A may be pre-existing inside band 1602, which may be implemented as band 706. In other embodiments, the first adhesive component 1604A may be coated onto the inside of the walls of band 1602 or incorporated into the material of which the band 1602 is composed such that the first adhesive component 1604A is pre-filled inside band 1602. In some embodiments, as shown in FIG. 16B, a second adhesive component 1604B may be inserted through a catheter 1606 after band 1602 is positioned around the papillary muscles, such as papillary muscles 702 and 704, and is ready to be hardened.

Additionally or alternatively, a heat sensitive adhesive may be used, and the temperature of the adhesive may be controlled within catheter 1606 to avoid the adhesive hardening in catheter 1606. Additionally or alternatively, the adhesive may be a light curing adhesive, and a fiber optic cable in catheter 1606 may be used to illuminate and harden the adhesive after the adhesive has been inserted into band 1602.

While FIGS. 7A-7C illustrate a circular band 706 placed around the papillary muscles 702, 704, in some embodiments, band 706 may include a wire that is configured to be bent to apposition the papillary muscles 702, 704. Additionally or alternatively, band 706 may include a wire in the form of a spiral that is configured to be inserted through a catheter, such as catheter 708, to make one or more full revolution(s) around the papillary muscles 702, 704.

In other embodiments, band 706 may be made of a shape-memory material, such as a shape-memory alloy, that is configured to lock band 706 into a pre-defined shape to apposition the papillary muscles 702, 704. By way of example, after insertion into ventricle 701 and placement around the papillary muscles 702, 704, the shape-memory material of band 706 may be heated to change the shape of band 706. Additionally or alternatively, the shape-memory material of band 706 may be cooled during insertion into ventricle 701 of the heart to maintain the shape of band 706. Afterwards, the shape-memory material of band 706 may heat up to body temperature to change the shape of band 706 into a pre-defined shape. In some embodiments, the shape-memory material of band 706 may be heated by passing electrical current through band 706 and/or by conductive heating from a heating element.

Disclosed embodiments may include any one of the following bullet-pointed features alone or in combination with one or more other bullet-pointed features, whether implemented as a method, device, or system.
A device for repositioning papillary muscles
a band configured to at least partially surround a cluster of papillary muscles, the cluster having an outer peripheral boundary, wherein the band comprises a first end and a second end.
a winch coupled to a location proximate the first end of the band, wherein the winch is configured to adjust a length of the band to apposition the papillary muscles in the cluster.
the winch is configured to pull the pullwire to adjust the length of the band.
the pullwire is coupled to a location proximate the second end of the band.
the band is configured to form a closed loop around the cluster of papillary muscles.
the winch is configured to adjust the length of the band to change a circumference of the closed loop.
the winch is configured to pull on the first end and the second end of the band to decrease the circumference of the closed loop.
the winch is configured to pull on strings coupled to the first end and the second of the band to decrease the circumference of the closed loop.
the band is configured to form an open C-shaped partial loop around the cluster of papillary muscles.
an adjustment of the winch is configured to change a shape of the band to apposition the papillary muscles in the cluster.
the band is configured to bend inward when the winch is adjusted, thereby appositioning the papillary muscles in the cluster.
the band comprises at least one of a flexible polymer, a cut metal tube, or a series of interconnected segments configured to rotate relative to each other.
a band configured to form a loop around a cluster of papillary muscles, the cluster having an outer peripheral boundary defined by the loop,
the band has a first end and a second end,
the first end and the second end of the band are configured to be twisted together to adjust a circumference of the loop formed by the band and lock the band at a predetermined circumference.
a band configured to be inserted into a ventricle of a heart to form a loop around a cluster of papillary muscles.
during insertion of the band into the ventricle of the heart, a shape of the band comprises a spiral shape configured to make at least one full revolution around the cluster of papillary muscles.
a shape memory alloy configured to modify the shape of the band after insertion of the band into the ventricle of the heart such that the band is configured to apposition the papillary muscles in the cluster.
the shape memory alloy is heated after insertion of the band into the ventricle of the heart to change the shape of the band.
the shape memory alloy is cooled during insertion of the band into the ventricle of the heart to maintain the shape of the band.
wherein the shape memory alloy is heated up to body temperature to change the shape of the band.
a band configured to form an open C-shaped partial loop around a cluster of papillary muscles, the cluster having an outer peripheral boundary partially defined by the partial loop,
the band is configured to be adjusted to change a shape of the band and apposition the papillary muscles in the cluster.
a pullwire coupled to an end of the band, wherein the pullwire is configured to be pulled to adjust the band.
a pullwire pulling mechanism.
a pullwire pulling mechanism is configured to be actuated by a rotation of a component of the pullwire pulling mechanism.
the pullwire pulling mechanism includes a screw.
a pullwire ratchet mechanism configured to allow the pullwire to pass through in only one direction.
a band configured to lock into a bent shape upon being adjusted such that when an adjustment force is released, the band is configured to maintain the bent shape.
a band includes a metal tube cut into a pattern, wherein the pattern forms latches configured to lock adjacent segments of the metal tube when the band is bent.
a band is configured to be locked into a geometric configuration at any time while the band is being adjusted.
a band is configured to be locked into the geometric configuration by filling the band with a material that hardens after insertion into the band.
a band includes a sealed inflatable tube.
a sealed inflatable tube is configured to form a predetermined shape when inflated.
a sealed inflatable tube is configured to be inflated after being passed around the papillary muscles,
a sealed inflatable tube is configured to form the predetermined shape after inflation to apposition the papillary muscles.
a band is configured to be locked when the sealed tube is inflated to maintain the appositioning of the papillary muscles.
a band is configured to be inflated with a material that hardens within the band, thereby maintaining an inflated shape to apposition the papillary muscles.

While the present disclosure is described herein with reference to illustrative embodiments of bands used for particular applications, such as for navigation through a ventricle of a heart for cardiac repair, it should be understood that the embodiments described herein are not limited thereto. Those having ordinary skill in the art and access to the teachings provided herein will recognize additional modifications, applications, embodiments, and substitution of equivalents that all fall within the scope of the disclosed embodiments. Accordingly, the disclosed embodiments are not to be considered as limited by the foregoing or following descriptions.

The many features and advantages of the present disclosure are apparent from the detailed specification, and thus it is intended by the appended claims to cover all such features and advantages of the present disclosure that fall within the true spirit and scope of the present disclosure. Further, since numerous modifications and variations will readily occur to those skilled in the art, it is not desired to limit the present disclosure to the exact construction and operation illustrated and described and accordingly, all suitable modifications and equivalents may be resorted to, falling within the scope of the present disclosure.

Moreover, those skilled in the art will appreciate that the conception upon which this disclosure is based may readily be used as a basis for designing other structures, methods, and systems for carrying out the several purposes of the present disclosure. Accordingly, the claims are not to be considered as limited by the foregoing description.

1. A device for repositioning papillary muscles, the device comprising:
a band configured to at least partially surround a cluster of papillary muscles, the cluster having an outer peripheral boundary, wherein the band includes a first end and a second end; and
a winch coupled to a location proximate the first end of the band, wherein the winch is configured to adjust a length of the band to apposition the papillary muscles in the cluster.

2. The device of claim 1, further comprising a pullwire, wherein the winch is configured to pull the pullwire to adjust the length of the band.

3. The device of claim 2, wherein the pullwire is coupled to a location proximate the second end of the band.

4. The device of claim 1, wherein the band is configured to form a closed loop around the cluster of papillary muscles, and wherein the winch is configured to adjust the length of the band to change a circumference of the closed loop.

5. The device of claim 4, wherein the winch is configured to pull on the first end and the second end of the band to decrease the circumference of the closed loop.

6. The device of claim 4, wherein the winch is configured to pull on strings coupled to the first end and the second end of the band to decrease the circumference of the closed loop.

7. The device of claim 1, wherein the band is configured to form an open C-shaped partial loop around the cluster of papillary muscles, and wherein an adjustment of the winch is configured to change a shape of the band to apposition the papillary muscles in the cluster.

8. The device of claim 7, wherein the band is configured to bend inward when the winch is adjusted, thereby appositioning the papillary muscles in the cluster.

9. The device of claim 8, wherein the band includes at least one of a flexible polymer, a cut metal tube, or a series of interconnected segments configured to rotate relative to each other.

10.-28. (canceled)
